(19) United States
(12) Patent Application Publication
(10) Pub. No.: US 2003/0096410 A1
(43) Pub. Date: May 22, 2003

(54) Methodology for constructing human / goat chimeras for the production of human cells

(76) Inventors: Fanyi Zeng, Gaithersburg, MD (US); Shu-zhen Huang, Gaithersburg, MD (US); Yi-Tao Zeng, Gaithersburg, MD (US)

Correspondence Address: Hung Chang Lin, 8 Schindler Court, Silver Spring, MD, 20903, US

(21) Appl. No.: 10/178,036

(22) Filed: Jun. 24, 2002

(30) Foreign Application Priority Data
Nov. 19, 2001 (CN) 01132231.4
Mar. 21, 2002 (CN) 02111124.3

(51) Int. Cl. C12N 5/08; A01N 1/02

(52) U.S. Cl. 435/372

(57) ABSTRACT

Fresh human hematopietic stem cells (HSC) obtained from umbilical cord blood, bone marrow, liver or other human tissues are directly injected into peritoneal cavity of fetal goat at 45-55 day gestation age under B-type ultrasonographic scan. Human HSC expands for up to 1000-10,000 fold in circulatory blood of recipient goats and the human HSC phenotype with non-differentiation remains stable up to over 10 months. The human cells can also grow and propagate in liver and other tissues of the recipient goats. The stored stem cells can be used for disease treatment, organ transplantation and in vivo assays of drugs.

### BACKGROUND OF THE INVENTION

(1) Field of the Invention

The invention relates to the production of stem cells, particularly to human/goat hematopietic stem cells.

(2) Brief Description of Related Art

In utero transplantation of hematopietic stem cells (HSC) into fetus for propagation, expansion and differentiation appears to be among the methodologies-of-choice for prenatal treatment on severe genetic diseases. Allogeneic HSC engraftment into recipient fetus at an early gestational stage can avoid immunoactive responses. This approach has been applied to transplantation of human HSC into fetal sheep and hematopoietic chimerism has been obtained. However, the methodology for producing human/sheep xenogenic chimeric models is traumatic and requires the induction of anethsthesia, followed by exposure of the fetus by maternal laparotomy and hysterotomy, disposing high risk of miscarriage. In utero transplantation of human HSC is carried out at 55-65 days of gestation age and this procedure is performed only once in each animal. Therefore, the engraftment rate is low.

### SUMMARY OF THE INVENTION

One objective of this invention is to provide a safe, non-surgical method for the transplantation of human stem cells to goat. Another objective of this invention is to reduce the risk of miscarriage during transplantation. Still another objective of this invention is to increase the transplantation rate of stem cells. A further objective of this invention is to increase the success rate of the transplantation of stem cells from human to goat.

This invention describes a methodology which is safe, reliable, with low miscarriage risk but high success transplantation rate for the production of human/goat chimeric model. Fresh human hematopietic stem cells (HSC) obtained from cord blood, bone marrow or other tissues were directly injected into the peritoneal cavity of fetal goats at as early as 45-55 day gestation age under B-type ultrasonographic scan. The identification results confirmed that chimerism exists in goat blood, liver and other organs. Human HSC expanded for up to 1000-10,000 fold in circulatory blood of the recipient goats and the human HSC phenotype, with a partial-differentiation remained stable up to 10 months or more. The human liver-like cells are evident in the transplanted goat liver tissues, characterized by morphology, physiology and expression of human serum albumin gene. The procedure of injection of human HSC could repeat many times with very few immunoactive responses. This invention established a novel technique platform for human HSC expansion, differentiation, storage as well as human specific gene expression in goat in vivo. The human/goat xenogenic chimeras may serve as a conventional resource for human hematopoietic stem cells/progenitors. In addition, it provides a potential way for prenatal therapy with in utero HSC transplantation as well as xenogenic organ transplantation.

### BRIEF DESCRIPTION OF THE FIGURES

FIG. 1A schematic presentation of the method for the injection of human stem cells into the peritoneal cavity of fetal goats under B-type ultrasonographic scan.

FIG. 2 FACS analysis of CD20⁺, CD34⁺, CD14⁺ and GPA⁺ cells in blood samples of the goats engrafted with human HSC.

FIG. 3 Amplification of human CD34 and GPA DNAs in the blood of engrafted goats by PCR analysis.

FIG. 4 RT-PCR analysis of CD34 and GPA mRNAs derived from human cells in blood of the engrafted goats.

FIG. 5 Southern blot analysis of the engraftment of human α-satellite DNA in goats. Genomic DNA was extracted from goats and then digested with Eco RI. The probe was p177H8 labeled with (α-³²P)dCTP (Amersham, UK). The positive hybridized band is 2.7 kb.

FIG. 6 Interphase FISH analysis of human Y-chromosome-positive cells in the circulating blood of recipient goats.

FIG. 7 A-upper: Positive nuclear PCNA staining result of normal human liver; B-middle:PCNA staining results of livers of transplanted goats; C-lower Negative nuclear PCNA staining result of normal goats.

FIG. 8 A-upper Positive cytoplasmic labeling of human liver with immuno-staining of human HSA; B-middle Positive HSA staining of livers of transplanted goats; C-lower Negative HAS labeling of normal goats.

FIG. 9 A-upperhHNF-β specific 368 base pair fragment in human and transplanted goat liver. No amplification was shown in normal goats liver and human blood cells; B-bottom hALB specific 400 base-pair fragment only in human and transplanted goat livers.

### DETAILED DESCRIPTION OF THE INVENTION

Prenatal diagnosis is useful for at-risk pregnancies of severe genetic diseases. However, in high risk pregnancies due to prevalent disease, prenatal diagnosis and termination of pregnancy impose undesirable physiological and psychological burdens to the parents, especially pregnant mothers who had more than one conception during their reproductive age. In such medical scenario prenatal treatment may provide the ultimate solution. Currently, in utero transplantation of hematopoetic stem cells (HSC) into fetus for propagation, expansion and differentiation appears to be among the methodologies-of-choice for prenatal treatment. Allogeneic HSC engraftment into recipient fetus in utero at a gestational stage prior to development and maturation of the immunological barrier in the recipient fetus could avoid immunoactive responses. This approach has been applied to transplantation of human HSC into normal and genetically defective mice. Hematopoietic chimerism and multilineage differentiation have also been obtained in sheep. However, the methodology for producing human/sheep xenogenic chimeric models is traumatic and requires the induction of anethsthesia, followed by exposure of the fetus by maternal laparotomy and hysterotomy, causing high risk of miscarrige. In utero transplantation of human HSC is carried out over 55-65 days of gestation age and this procedure is performed only once in each animal. Therefore, the engraftment rate is low.

This invention is to establish a safe, reliable with low miscarriage risk but high success transplantation rate methodology for production goat chimeras, in which the engraftment, expansion, differentiation, evolvement and plasticity of human HSC in goats are investigated at in vivo level.

The methodology that we used for producing human/goat chimeras is a non-surgical technique, described as follows

(1) Collection of human hematopoietic stem cells (HSC) from fresh umbilical cord blood, bone marrow or other tissues;

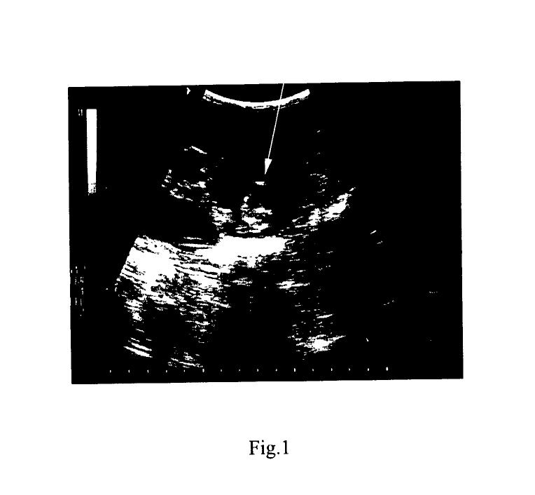

(2) The fresh human HSC is immediately injected into the peritoneal cavity of fetal goats at as early as 45-55 day gestation age under B-type ultrasonographic monitoring, but without the induction of anethsthesia, nor the exposure of the fetus by maternal laparotomy and hysterotomy (FIG. 1);

(3) This procedure can be repeated many times and obtain the following results:

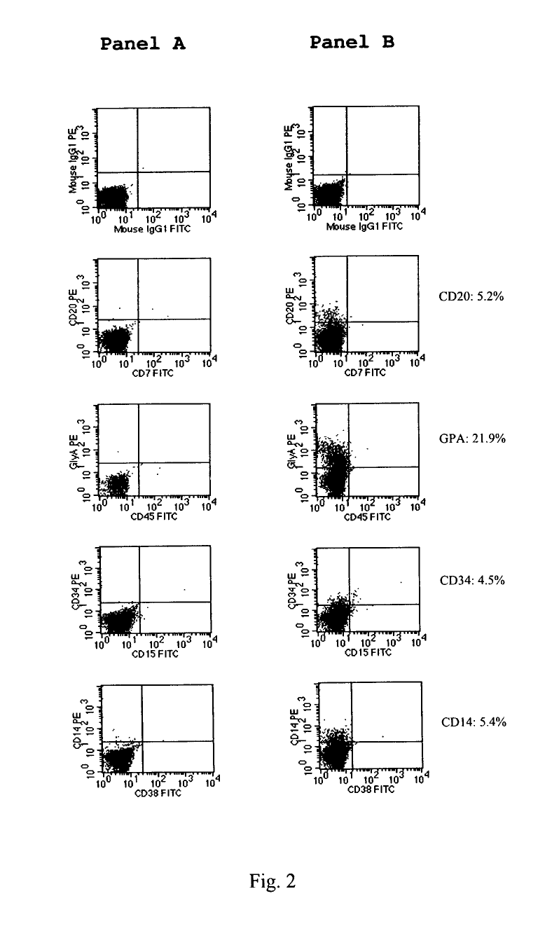

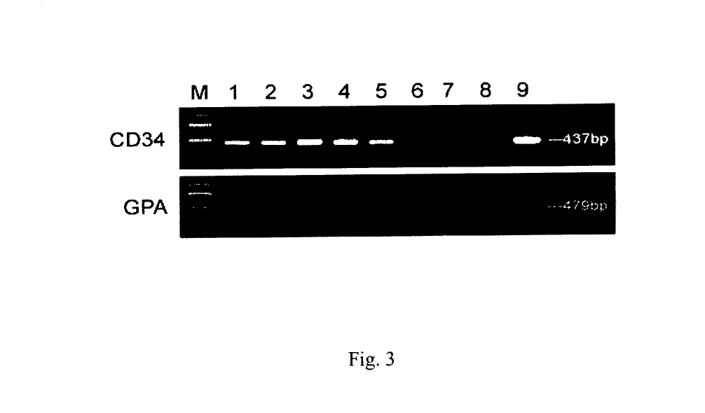

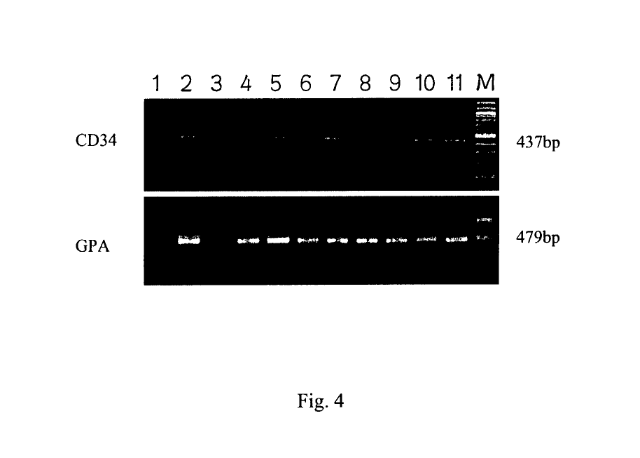

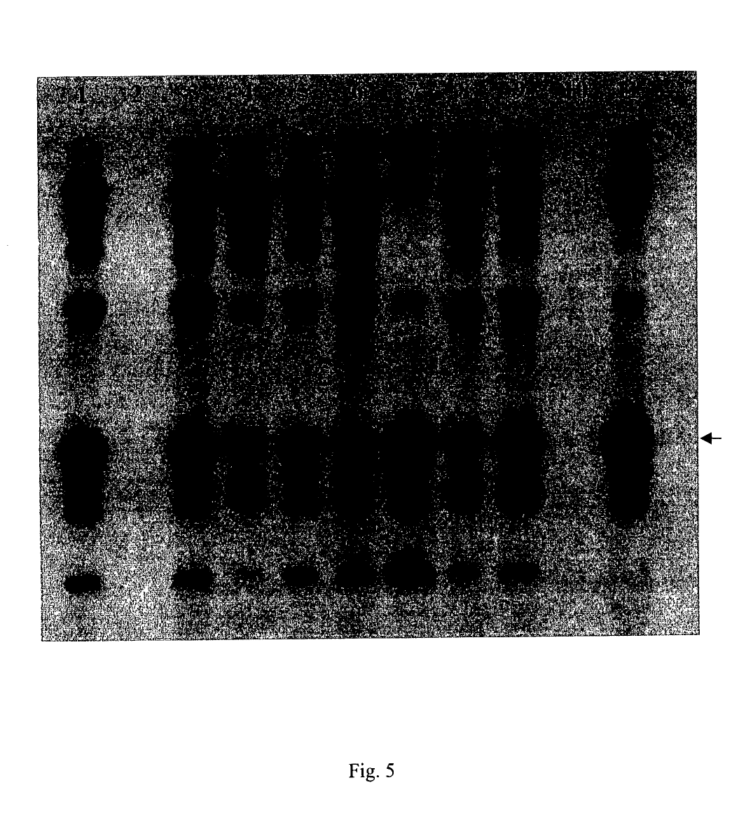

(i) The detection of the human/goat chimeric models by FACS analysis (FIG. 2) and molecular (CD34, GPA, etc) identification (PCR, RT-PCR and Southern blotting) by FACS analysis (FIG. 2) and molecular indicated:T he engraftment of human HSC (CD20⁺, CD34⁺, CD14⁺ and GPA⁺ cells) in recipient goats indeed occured (as seen in FIG. 3-5.)

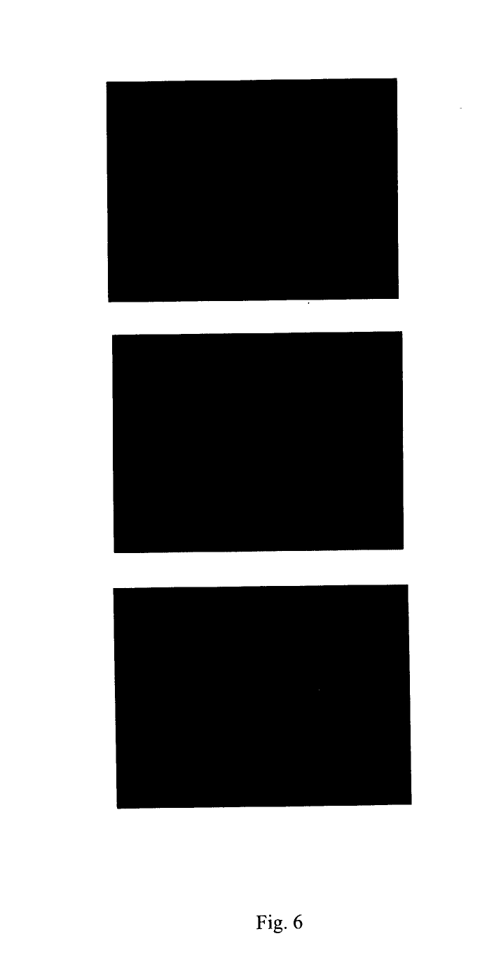

(ii) The interphase FISH analysis showed that human Y-chromosome-positive cells existed in the circulating blood of recipient goats as seen in FIG. 6. The top picture (A-upper) shows that normal male has positive hybridized signal with each of the blood cells, the middle picture (B-middle) shows that blood cells of normal female goat do not produce hybridized signals in their blood cells. The bottom picture (C-lower) shows that female goat transplanted with male cord cells with one blood cell at interphase produced a positive hybridized signal.

(iii) Human HSC in goat circulatory blood expands for up to 1000-10000 fold;

(iv) The human HSC with a partial-differentiation phenotype in recipient goats remains stable for over 10 months.

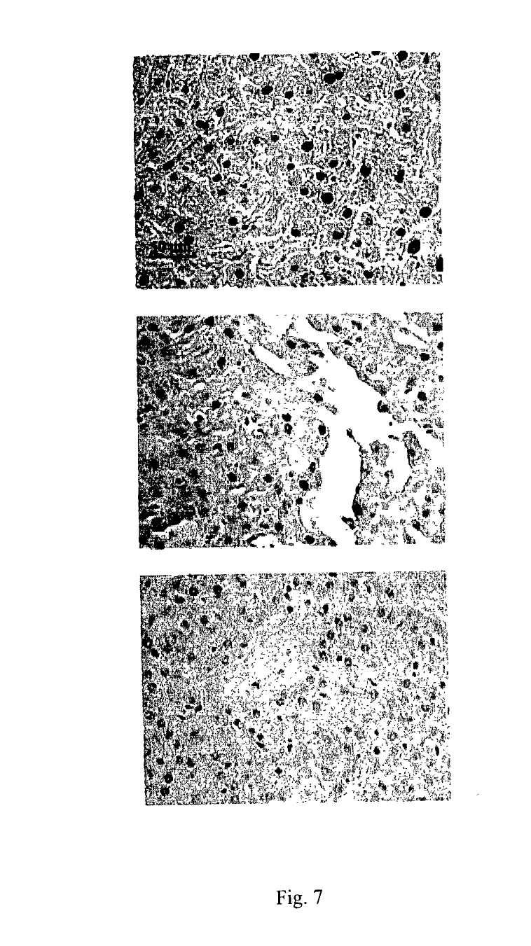

Meanwhile, human/goat chimeric liver was also obtained with the production of human/goat xenogenic chimeras. Four human HSC-transplanted goats, all with detectable human-like cells in the blood circulation, were sacrificed. The liver specimens were collected and subjected to immunochemistry, FACS, FISH analyses as well as RT-PCR detection of human hepatocyte nuclear factor (hHNF)-3β and human serum albumin (hALB) mRNAs. The results indicate that the human liver-like cells are chimeric in goat liver tissues, characterized by morphology, physiology and expression of human specific genes. (FIG. 7,8,9)

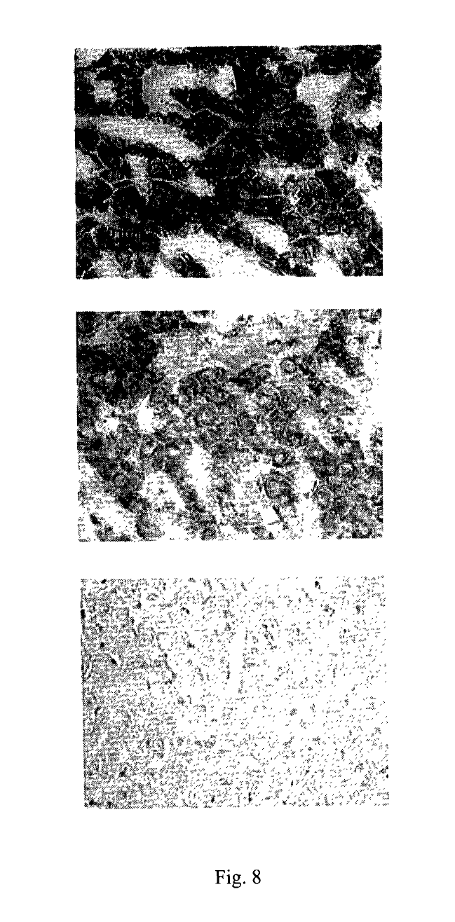

Typical staining results are shown in FIG. 7,8. Human liver cells showed positive nuclear PCNA staining (FIG. 7A-upper), which was also detected in liver sections of all four transplanted goats (FIG. 7B-middle) but all five normal goats were negative for PCNA (FIG. 7C-lower). Immunostaining of human HSA showed positive cytoplasmic labeling in human liver (FIG. 8A-upper) and also livers from the transplanted goats (FIG. 8B-middle). None of the 5 normal goats had positive HSA staining in any liver sections (FIG. 8C-lower).

In FACS analysis, human HSA was abundantly expressed in liver cells obtained from transplanted goats but not detectable in normal ones. From approximately 10⁵ cells that we have examined, 49.7% of liver cells showed positive expression of human HSA in a chimeric goat.

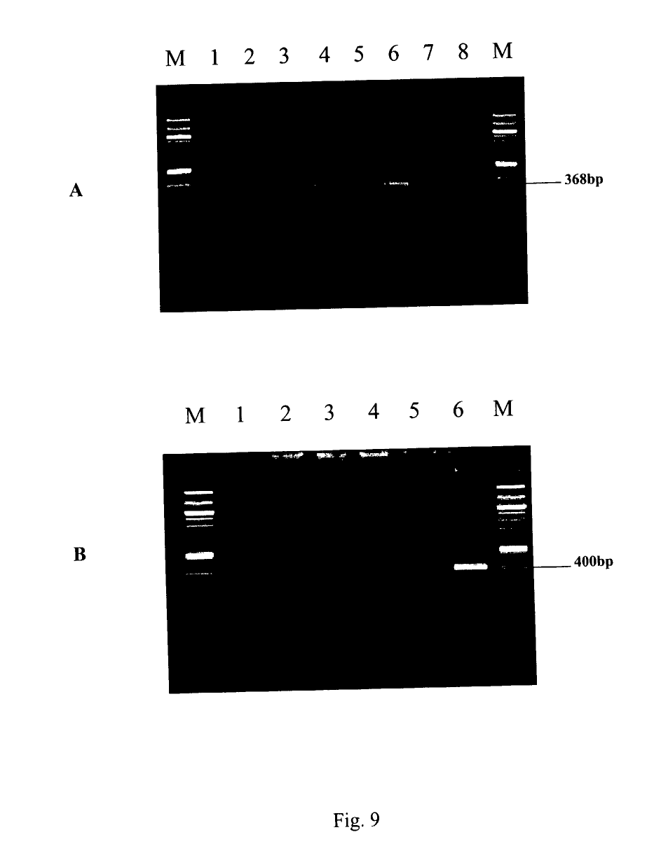

In human liver specific gene expression, FIG. 9 shows hHNF-3β and hALB mRNA expression in liver tissues of human and transplanted goats but not in liver tissue of the normal goats. RT-PCR revealed the presence of hHNF-3β specific 368 base-pair fragment in human and transplanted goat livers. No amplification was shown in liver tissue of normal goats and human blood cells (FIG. 9A-upper). Similar results for RT-PCR analysis of hALB mRNA were obtained: only the specific 400 base-pair fragment were found in human and transplanted goat livers (FIG. 9B-bottom)

The novelty of this invention is:

(1) Goats are firstly used to be as a large animal for the engraftment, expansion, differentiation, evolvement of human HSC at an in vivo level;

(2) As compared to sheep, goats provide an appropriate body mass and size. Its multi-fetal pregnancy and life-span are manageable in such experimental model;

(3) The Chinese goats that are used have been farmed in the local area for thousands of years and are therefore easy to be raised;

(4) The methodology is safe, reliable and non- traumatic with low miscarriage risk but high success transplantation rate for production goat chimerism, in which the whole procedure of human HSC transplantation into fetal goats is monitored under B-scan ultrasonographic scan. The human HSC obtained from fresh cord blood, bone marrow and other tissues are immediately injected into the peritoneal cavity of fetal goats. Significantly, the human HSC injection moves ahead to as early as 45 days of gestation age. Therefore, immunoactive response is greatly decreased. Moreover, the above injection procedure can be repeated several times with minimum number of cells which avoids side reaction caused by a single injection of a large dosage of cells,thus leads to a high success rate for the production of chimeric goats.

(5) The mechanism of Human HSC/progenitor differentiation and development is firstly demonstrated at goat in vivo level;

(6) Embryology, histology, immunology, cell and molecular biological techniques are used for systemic study of human HSC differentiation, development and evolvement in chimeric goats;

(7) The expression of human specific genes is firstly studied in circulatory blood and liver tissues of the chimeric goats.

In general, this invention describes the morphological, immunological and physiological characteristic of human liver-like cells derived from human HSC under the goat inner circumstances. The invention confirms that the genes of human liver-like cells that grow and propagate in recipient goat liver tissues have specific expression functions. This invention sets up a novel technique platform for human HSC expansion, differentiation, storage as well as human specific gene expression in goat circulatory blood, liver and other organs. The human/goat xenogenic chimeras may also serve as a conventional resource for human hematopoietic stem cells/progenitors. In addition, it provides a potential way for prenatal therapy with the method of in utero HSC transplantation as well as for xenogenic organ transplantation and in vivo screening assays for chemo-agents.

While the invention is described for the transplantation of stem cells from human to goats, it is conceivable that the technique is not limited to human/goat relationship; nor is it limited to the transplantation of liver cells. While the preferred embodiments have been described, it will be apparent to those skilled in the art that various modifications may be made in the embodiments without departing from the spirit of the present invention. Such modifications are all within the scope of this invention.

1. A method of transplanting stem cells from one body to a second body, comprising the steps of:
obtaining stem cells of first tissue from said first body;
aging said stem cells after gestation;
injecting in vivo said stem cells into a part of a second body;
propagating said stem cells in said second body; and
storing said stem cells for treating genetic diseases.

2. The method of transplanting as described in claim 1, wherein said first body is human and said second body is a goat.

3. The method of transplanting as described in claim 1, wherein said stem cells are human hematopoetic stem cells.

4. The method of transplanting as described in claim 1, wherein said first tissue is the tissue of an umbilical cord.

5. The method of transplanting as described in claim 1, wherein said first tissue is the tissue of bone marrow.

6. The method of transplanting as described in claim 1, wherein said first tissue is the tissue of liver.

7. The method of transplanting as described in claim 2, wherein the age of gestation takes 45-55 days.

8. The method of transplanting as described in claim 1, wherein said cells propagate 1000-10,000 fold.

9. The method of transplanting as described in 2, wherein said stem cells are injected into the peritoneal cavity of the goat under B-type ultrasonographic scan but without surgical procedure.
